(19) United States
(12) Patent Application Publication
(10) Pub. No.: US 2015/0164630 A1
(43) Pub. Date: Jun. 18, 2015

(54) FILTER SUPPORT MEMBERS

(71) Applicants: Eric JOHNSON, Woodside, CA (US); Jeremy STIGALL, Carlsbad, CA (US)

(72) Inventors: Eric JOHNSON, Woodside, CA (US); Jeremy STIGALL, Carlsbad, CA (US)

(21) Appl. No.: 14/574,203

(22) Filed: Dec. 17, 2014

Related U.S. Application Data
(60) Provisional application No. 61/917,865, filed on Dec. 18, 2013.

(51) Int. Cl. A61F 2/01

(52) U.S. Cl. CPC A61F 2/01; A61F 2002/016; A61F 2210/0014; A61F 2230/0008; A61F 2002/011

(57) ABSTRACT

An endoluminal filter comprising a first support member having a first end and a second end forming a first semi-circular structure and a second support member having a first end and a second end, the second support member forming a second semi-circular structure, the first end and second end of the second support structure attached to the first end and second end of the first support member. The filter also includes a material capture structure extending within an internal area of the first semi-circular structure and a retrieval feature. An endoluminal filter with a first support member and a second support member with the first support member and/or second support member configured in a loop structure. Methods for deploying the endoluminal filters disclosed herein within a lumen are also provided.

### CROSS REFERENCE TO RELATED APPLICATIONS

This application claims priority to application U.S. Provisional Patent Application No. 61/917,865, filed on Dec. 18, 2013, and titled “FILTER SUPPORT MEMBERS,” which is herein incorporated by reference in its entirety.

This application may be related to U.S. patent application Ser. No. 11/969,827, filed on Jan. 4, 2008, titled “ENDOLUMINAL FILTER WITH FIXATION,” and published as U.S. Patent Application Publication No. 2008/0147111 and U.S. Provisional Patent Application No. 62/090,580, filed on Dec. 11, 2014, and tilted “ENDOLUMINAL FILTER DESIGN VARIATIONS,” each of which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

This invention relates generally to devices and methods for providing filtration of debris within a body lumen. More particularly, the invention provides a retrievable filter placed percutaneously in the vasculature of a patient to prevent passage of emboli. Additionally, embodiments of the invention provide a filter that can be atraumatically positioned and subsequently removed percutaneously from a blood vessel.

### BACKGROUND

Embolic protection is utilized throughout the vasculature to prevent the potentially fatal passage of embolic material in the bloodstream to smaller vessels where it can obstruct blood flow. The dislodgement of embolic material is often associated with procedures which open blood vessels to restore natural blood flow such as stenting, angioplasty, arthrectomy, endarterectomy or thrombectomy. Used as an adjunct to these procedures, embolic protection devices trap debris and provide a means for removal for the body.

One widely used embolic protection application is the placement of filtration means in the vena cava. Vena cava filters (VCF) prevent the passage of thrombus from the deep veins of the legs into the blood stream and ultimately to the lungs. This condition is known as deep vein thrombosis (DVT), which can cause a potentially fatal condition known as pulmonary embolism (PE).

The first surgical treatment for PE, performed by John Hunter in 1874, was femoral vein ligation. The next major advancement, introduced in the 1950's, was the practice of compartmentalizing of the vena cava using clips, suture or staples. While effective at preventing PE, these methods were associated with significant mortality and morbidity (see, e.g., Kinney TB, Update on inferior vena cava filters, JVIR 2003; 14:425-440, incorporated herein by reference).

A major improvement in PE treatment, in which venous blood flow was maintained, was presented by DeWesse in 1955. This method was called the “harp-string” filter, as represented in FIG. 1A and FIG. 1B, in which strands of silk suture 12 were sewn across the vena cava 11 in a tangential plane below the renal veins 13 to trap thrombus. Reported clinical results demonstrated the effectiveness of this method in preventing PE and maintaining caval patency. (see, e.g., DeWeese M S, A vena cava filter for the prevention of pulmonary embolism, Arch of Surg 1963; 86:852-868, incorporated herein by reference). Operative mortality associated with all of these surgical treatments remained high and therefore limited their applicability.

The current generation of inferior vena cava (IVC) filters began in 1967 with the introduction of the Mobin-Uddin umbrella 21 (FIG. 1C) which is described in further detail in U.S. Pat. No. 3,540,431. The Greenfield filter (FIG. 1D) was introduced in 1973 and is described in further detail in U.S. Pat. No. 3,952,747. These conical-shaped devices were placed endoluminaly in the IVC and utilized hooks or barbs 20, 30 to pierce the IVC wall and fix the position of the device. A variety of conical-shaped, percutaneously placed vena cava filters, based upon this concept are now available. For example, the TULIP with a filter structure 41 (FIG. 1E) further described in U.S. Pat. No. 5,133,733; the RECOVERY with a filter structure 51 (FIG. 1F) further described in U.S. Pat. No. 6,258,026; and the TRAPESE with a filter structure 61 (FIG. 1G) further described in U.S. Pat. No. 6,443,972.

The next advancement in filters added the element of recoverability. Retrievable filters were designed to allow removal from the patient subsequent to initial placement. Retrievable filters are generally effective at preventing PE yet they have a number of shortcomings, such as, for example: failure of the device to deploy into the vessel properly, migration, perforation of the vessel wall, support structure fracture, retrievability actually limited to specific circumstances, and formation of thrombosis on or about the device.

Problems associated with retrievable, conical-shaped devices, such as those illustrated in FIG. 1D, FIG. 1E and FIG. 1F, have been reported in the medical literature. These reported problems include tilting which makes it difficult to recapture the device and compromises filtration capacity. Hooks 30, 40, 50, 60 used to secure these devices have been reported to perforate the vessel wall, cause delivery complications, and fracture. A partially retrievable system is described in detail in pending U.S. Pat. No. 2004/0186512 (FIG. 1H). In this system, the filter portion 71 can be removed from the support structure 70, but the support structure remains in-vivo. All of these described devices share the common limitation that they can be retrieved from only one end. Each of the above referenced articles, patents and patent applications are incorporated herein in its entirety.

Additional retrievable endoluminal filters are disclosed in US 2008/0147111 to Eric Johnson et al. (FIG. 1I). FIG. 1I is a perspective view of an endoluminal filter 89 having a first support member 90 having a first end and a second end and a second support member 91 attached to the first end of the first support member 90 or the second end of the first support member 91. The second support member 91 forms a crossover 92 with the first support member 90. The second support member 91 and the first support member 90 are movable relative to each other at the crossover point. The first support member 90 and second support member 91 are moveable relative to one another at crossover 92. A material capture structure 93 extends between the first and second support members 90, 91, the crossover 92 and the first end or the second end of the first support member 90. The filter illustrated in FIG. 1I has a retrieval feature 94 on the first end and a retrieval feature 94 on the second end. A tissue anchor 95 projects from the first support member 90 and the second support member 91. The tissue anchor 95 can improve engagement with a luminal wall. The first support member 90 and second support member 91 can be joined together with a crimp 96.

FIG. 1J illustrates an endoluminal filter deployed in the superior vena cava.

In view of the many shortcomings and challenges that remain in the field of endoluminal filtering, there remains a need for improved retrievable endoluminal filters.

### SUMMARY OF THE DISCLOSURE

The present invention relates to endoluminal filters and methods for deploying and retrieving endoluminal filters.

In any of the embodiments disclosed herein an endoluminal filter, including a first support member having a first end and a second end; a second support member having a first end and a second end, the second support member connected to the first support member; a material capture structure extending within an internal area of the first support member; and a retrieval feature engaged with the first support member or second support member.

In any of the embodiments disclosed herein an endoluminal filter can further include at least one tissue anchor on the first support member or the second support member.

In any of the embodiments disclosed herein the retrieval feature can be formed on the surface of the filter adjacent to an attachment between the first semi-circular structure and second semi-circular structure.

In any of the embodiments disclosed herein the first support member and the second support member can be formed from a single wire.

In any of the embodiments disclosed herein the first support member and second support member can be made out of a shape memory material.

In any of the embodiments disclosed herein the first support member and second support member can have smooth surfaces.

In any of the embodiments disclosed herein the first support member can form a first semi-circular structure, the second support member can form a second semi-circular structure, and the first end and second end of the second support structure can be attached to the first end and second end of the first support member.

In any of the embodiments disclosed herein the first support member can have a sinusoidal pattern forming the first semi-circular structure and/or the second support member can have a sinusoidal pattern forming the second semi-circular structure.

In any of the embodiments disclosed herein the first support member and/or second support member can include a plurality of inflection points or bends.

The first semi-circular structure and/or second semi-circular structure can have an elliptical shape.

In any of the embodiments disclosed herein the first support member and second support member can form an acute angle at each of an intersection between the ends of the first support member and second support member.

In any of the embodiments disclosed herein the first support member can form a first loop structure.

In any of the embodiments disclosed herein the first support member can further include a movable portion.

In any of the embodiments disclosed herein the second support member can form a second loop.

In any of the embodiments disclosed herein the second support member can further include a movable portion.

In any of the embodiments disclosed herein the second support member can be attached to the first support member.

In any of the embodiments disclosed herein an endoluminal filter can further include a third support member having a first end attached to the first loop structure and a second end attached to the second loop structure.

In any of the embodiments disclosed herein the third support member can have a coiled structure.

In any of the embodiments disclosed herein the retrieval feature can be located where the third support member attaches to the first loop structure or the second loop structure.

In any of the embodiments disclosed herein an endoluminal filter can further include a third loop structure and a fourth support member having a first end attached to the third loop structure and a second end attached to the second loop structure.

In any of the embodiments disclosed herein the second support member can include a semi-circular structure.

In any of the embodiments disclosed herein the second support member can have a sinusoidal pattern forming the semi-circular structure or a plurality of bends or inflection points.

In any of the embodiments disclosed herein the first support member can have a sinusoidal pattern in the first loop structure or a plurality of bends or inflection points in the first loop structure.

In any of the embodiments disclosed herein the first support member can form a first semi-circular structure, the second support member forming a second semi-circular structure, the first support member attached to the second support member at a portion besides the first end or second end of the first support member or second support.

In any of the embodiments disclosed herein an endoluminal filter can further include a third support member having a first end attached to the first semi-circular structure and a second end attached to the second semi-circular structure.

In any of the embodiments disclosed herein the third support member can have a coiled structure.

In any of the embodiments disclosed herein the first support member can have a sinusoidal pattern in the first semi-circular structure or a plurality of bends or inflection points in the first semi-circular structure.

In any of the embodiments disclosed herein a method of positioning a filter within a lumen, including: advancing a sheath containing the filter of claim 1 through the lumen; deploying a portion of the filter of claim 1 from the sheath into the lumen to engage the lumen wall while maintaining substantially all of the material capture of the filter within the sheath; and deploying the material capture structure of the filter of claim 1 from the sheath to a position across the lumen.

In any of the embodiments disclosed herein the method can further include maneuvering a snare towards the filter in the same direction used during the advancing step; and engaging the snare with a filter retrieval feature positioned against a wall of the lumen.

In any of the embodiments disclosed herein the method can further include maneuvering a snare towards the filter in the opposite direction used during the advancing step; and engaging the snare with a filter retrieval feature positioned against a wall of the lumen.

In any of the embodiments disclosed herein the method can further include deploying the filter retrieval feature from the sheath after the deploying the material capture structure step.

In any of the embodiments disclosed herein the method can further include deploying a filter retrieval feature from the sheath before the deploying the material capture structure step.

In any of the embodiments disclosed herein the deploying a portion of the filter step can further include engaging the lumen wall with a radial force generated by the filter.

In some embodiments endoluminal filters are provided. Endoluminal filters are provided including a first support member having a first end and a second end forming a first semi-circular structure, a second support member having a first end and a second end, a material capture structure extending within an internal area of the first semi-circular structure, and a retrieval feature. The second support member can form a second semi-circular structure. The first end and second end of the second support structure can be attached to the first end and second end of the first support member.

In any of the embodiments disclosed herein the filters can further include at least one tissue anchor on the first support member or the second support member. In any of the embodiments disclosed herein the at least one tissue anchor is formed on a surface of the first support member or the second support member.

In any of the embodiments disclosed herein the retrieval feature is formed on a surface of the first support member or the second support member. In any of the embodiments disclosed herein the filters the retrieval feature is formed on the surface of the filter adjacent to an attachment between the first semi-circular structure and second semi-circular structure.

In any of the embodiments disclosed herein the first support member and the second support member are formed from a single wire.

In any of the embodiments disclosed herein the first support member and second support member are made out of a shape memory material.

In any of the embodiments disclosed herein the first support member and second support member have smooth surfaces.

In any of the embodiments disclosed herein the first support member has a sinusoidal pattern forming the first semi-circular structure. In any of the embodiments disclosed herein the second support member has a sinusoidal pattern forming the second semi-circular structure.

In any of the embodiments disclosed herein the first support member includes a plurality of inflection points or bends. In any of the embodiments disclosed herein the second support member includes a plurality of inflection points or bends.

In any of the embodiments disclosed herein the first semi-circular structure has an elliptical shape. In any of the embodiments disclosed herein the second semi-circular structure has an elliptical shape.

In any of the embodiments disclosed herein the first support member and second support member do not form a crossover.

In any of the embodiments disclosed herein the first support member and second support member do not have a spiral structure.

In some embodiments endoluminal filters are provided herein. The endoluminal filters include a first support member forming a first loop structure, a second support member having a first end and a second end, a material capture structure extending in an area within the first loop structure, and a retrieval feature.

In any of the embodiments disclosed herein the filters further comprise at least one tissue anchor on the first support member or the second support member.

In any of the embodiments disclosed herein the second support member forms a second loop. In any of the embodiments disclosed herein the second support member is attached to the first support member. In any of the embodiments disclosed herein the second support member is crimped to the first support member.

In any of the embodiments disclosed herein the filters further comprise a third support member having a first end attached to the first loop structure and a second end attached to the second loop structure. In any of the embodiments disclosed herein the third support member has a coiled structure. In any of the embodiments disclosed herein the retrieval feature is located where the third support member attaches to the first loop structure or the second loop structure.

In any of the embodiments disclosed herein the filters further comprise a third loop structure and a fourth support member having a first end attached to the third loop structure and a second end attached to the second loop structure.

In any of the embodiments disclosed herein the second support member comprises a semi-circular structure. In any of the embodiments disclosed herein the second support member has a sinusoidal pattern forming the semi-circular structure. In any of the embodiments disclosed herein the second support member includes a plurality of bends or inflection points.

In any of the embodiments disclosed herein the first support member has a plurality of bends or inflection points in the first loop structure.

In any of the embodiments disclosed herein the first support member has a sinusoidal pattern in the first loop structure.

In any of the embodiments disclosed herein the retrieval feature is attached to the first loop structure.

In any of the embodiments disclosed herein the first support member and second support member do not crossover each other.

In any of the embodiments disclosed herein the at least one tissue anchor is formed on the surface of the first support member or the second support member.

In any of the embodiments disclosed herein wherein the first support member and second support member do not have a spiral structure.

In any of the embodiments disclosed herein the first support member further comprises a movable portion. In any of the embodiments disclosed herein the second support member further comprises a movable portion.

In some embodiments endoluminal filters are provided. The endoluminal filters can include a first support member having a first end and a second end forming a first semi-circular structure, a second support member having a first end and a second end forming a second semi-circular structure, the first support member attached to the second support member at a portion besides the first end or second end of the first support member or second support, a material capture structure extending within an internal area of the first semi-circular structure; and a retrieval feature.

In any of the embodiments disclosed herein the filters further comprise a third support member having a first end attached to the first semi-circular structure and a second end attached to the second semi-circular structure. In any of the embodiments disclosed herein the third support member has a coiled structure. In any of the embodiments disclosed herein the retrieval feature is located where the third support member attaches to the first semi-circular structure or the second semi-circular structure.

In any of the embodiments disclosed herein the first support member is joined to the second support member.

In any of the embodiments disclosed herein the at least one tissue anchor is formed on the surface of the first support member or the second support member.

In any of the embodiments disclosed herein the first support member has a sinusoidal pattern in the first semi-circular structure.

In any of the embodiments disclosed herein the first support member has a plurality of bends or inflection points in the first semi-circular structure.

In any of the embodiments disclosed herein the retrieval feature is attached to the first semi-circular structure.

In any of the embodiments disclosed herein the first support member and second support member do not have a spiral structure.

In some embodiments methods of positioning a filter within a lumen are provided. The methods include advancing a sheath containing any of the filters disclosed herein through the lumen, deploying a portion of the filter of any of the preceding claims from the sheath into the lumen to engage the lumen wall while maintaining substantially all of the material capture of the filter within the sheath, and deploying the material capture structure of the filter from the sheath to a position across the lumen.

In any of the embodiments disclosed herein the methods further include maneuvering a snare towards the filter in the same direction used during the advancing step and engaging the snare with a filter retrieval feature positioned against a wall of the lumen.

In any of the embodiments disclosed herein the methods further include maneuvering a snare towards the filter in the opposite direction used during the advancing step, and engaging the snare with a filter retrieval feature positioned against a wall of the lumen.

In any of the embodiments disclosed herein the methods further include deploying the filter retrieval feature from the sheath after the deploying the material capture structure step.

In any of the embodiments disclosed herein the methods further include deploying a filter retrieval feature from the sheath before the deploying the material capture structure step.

In any of the embodiments disclosed herein the methods further include engaging the lumen wall with the tissue anchor attached to the filter.

In any of the embodiments disclosed herein the methods further include engaging the lumen wall with a radial force generated by the filter.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-1J illustrate various prior art filters.

FIGS. 2A-2B illustrate various features of endoluminal filter devices.

FIGS. 3A-3B illustrate various features of endoluminal filter devices.

FIGS. 4A-4B illustrate various features of endoluminal filter devices.

FIGS. 5A-5B illustrate various features of endoluminal filter devices.

FIGS. 6A-6B illustrate various features of endoluminal filter devices.

FIGS. 7A-7B illustrate various features of endoluminal filter devices.

FIGS. 8A-8B illustrate various features of endoluminal filter devices.

FIGS. 9-22 illustrate various configurations of endoluminal filter devices.

FIGS. 23A-34F illustrate several alternative filtering structures.

### DETAILED DESCRIPTION

There remains a clinical need for improved endoluminal filter devices and methods. Improved endoluminal filter devices provide effective filtration over a range of lumen sizes and are easy to deploy into and retrieve from a lumen. The filters disclosed herein offer improved filters with a simpler construction design than prior art filters along with improved filtration properties.

Improved endoluminal filter devices minimize thrombosis formation or tissue ingrowth on the device and are resistant to migration along the lumen. Embodiments of the filter devices disclosed herein provide many improved features without the drawbacks of some of the prior art filters. The endoluminal filters disclosed herein have a number of uses such but are not limited to: embolic protection, thrombectomy, vessel occlusion, and tethered or untethered distal protection.

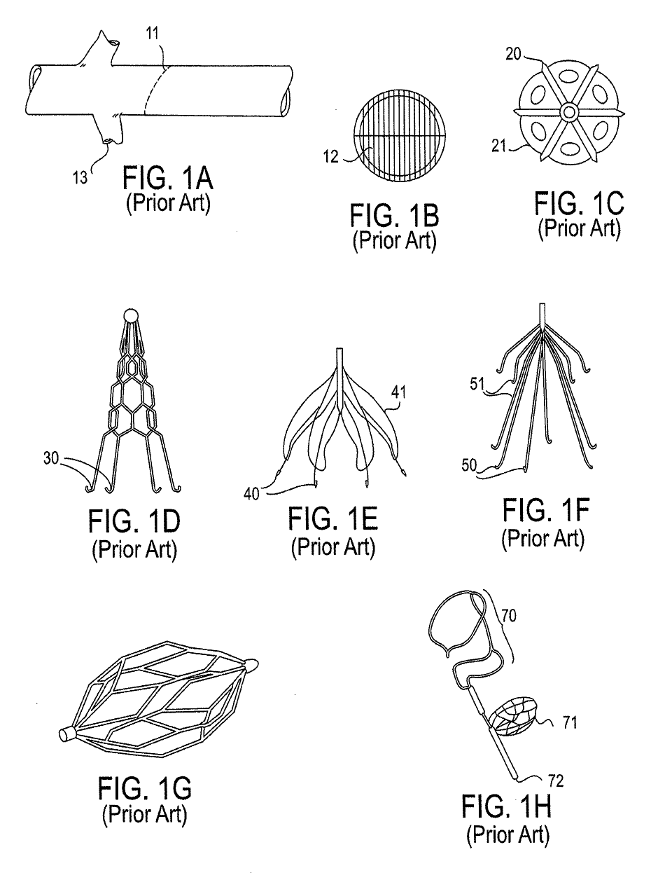

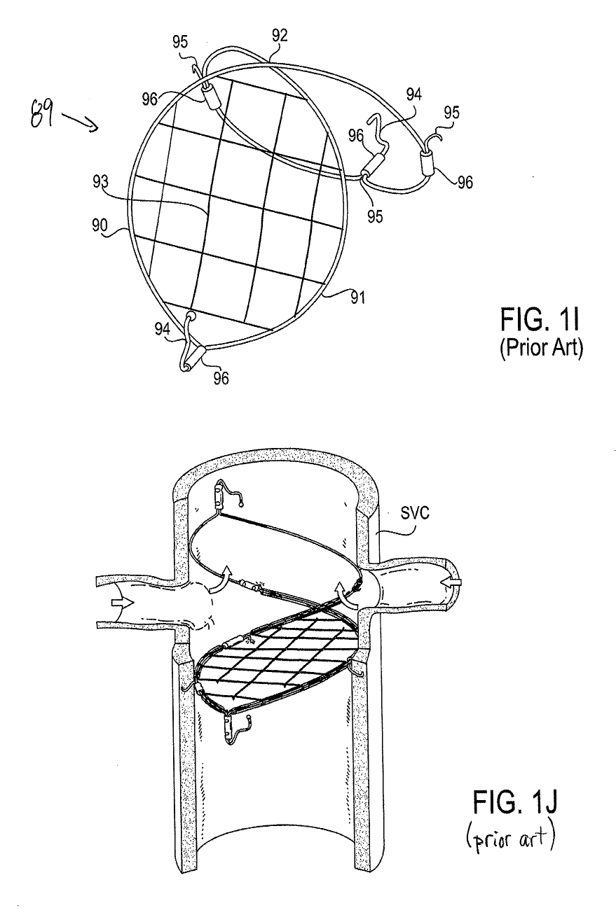

Some embodiments of filters disclosed herein do not have spiral support members, in contrast to the prior art filter illustrated in FIG. 1I which has support members 90, 91 that spiral in opposing directions. The filter illustrated in FIG. 1I, when viewed along the axis of the lumen in which it is implanted, has a support member that curves from a first position (e.g. 6 o'clock) at a first end longitudinally along the luminal wall to a second position at the second end that is approximately the same radial position (e.g. 6 o'clock) as the first position. The non-spiral arrangements of the support members disclosed herein can include arcuate structures that make less than a full rotation while transiting along the longitudinal dimension of the device.

The non-spiral arrangement of the support members can provide consistent and improved filtration with a less complicated structure than prior art filters. The filters having non-spiral arrangements disclosed herein have a shorter deployment length than prior art filters. In addition, the filters having non-spiral arrangements disclosed herein can be deployed with greater accuracy than prior art filters. Another benefit is that the physician may have greater experience and skill placing filters with non-spiral arrangements.

In some embodiments filters are disclosed herein including a first and second support member that form two semi-circular loops that are connected together. The semi-circular loops can be joined together from separate materials or made out of a single material, such as a wire. The semi-circular loops do not form a full circle and do not cross over the other support member. A material capture structure is formed across either or both of the semi-circular loop structures. The filter design offers improved uniform filtering with a simple and flexible design that maintains apposition with the lumen wall.

In some embodiments filters are disclosed herein with a first closed loop structure and an additional support structure. A material capture structure is formed across the first closed loop structure. In some embodiments the additional support structure includes a semi-circular or arched structure configured to engage a lumen wall. In some embodiments the additional support structure includes a second closed loop structure directly attached to the first loop structure or attached by an intervening support member. In another alternative the additional support structure can include a second closed loop structure directly or indirectly attached to the first closed loop structure and a third closed loop structure directly or indirectly attached to the second closed loop structure. In another alternative the additional support structure can have a coiled configuration.

In some embodiments filters are disclosed herein with a first support member forming an arched or semi-circular shape and a second support member forming an arched or semicircular shape that is attached directly or indirectly to the first support member. A material capture structure can extend between either or both of the arched or semi-circular shaped support members. The open ends of the first and second support members offer improved flexibility and engagement with the lumen wall when the filter is implanted in the patient.

The support members disclosed herein can have different shapes and textures. In some embodiments the support members can have a smooth texture. In some embodiments the support members can have a roughened texture, such as small projections or a jagged exterior, which can contact the vessel wall to hold the filter in place.

In some embodiments the support members can be modified to include filter attachment points. For example the support members can have a hole, hook, loop, divot, or other structure to facilitate holding a filter element or part of a material capture structure.

In some embodiments the support members disclosed herein can be patterned or shaped to provide filter attachment points. For example, the filter support members can have a curved or sinusoidal arrangement that can be used as filter attachment points.

Several embodiments provide improved filtration devices that are durable, provide effective and nearly constant filter capacity over a range of lumen sizes and are easily delivered and removed from a lumen. Additionally, embodiments can be delivered into and retrieved from a lumen using minimally invasive surgical techniques approaching either end of the filter.

In some embodiments the support frame is made using a shape memory material. The shape memory material may have a pre-shaped form that ensures the support elements are uniformly collapsible and, when deployed, provides a pre-defined range of controllable force against the lumen wall without use of hooks or barbs.

In some embodiments the filter frames disclosed herein can include at least one anchor or fixation point to promote atraumatic contact with the lumen walls. In some embodiments textured surfaces (e.g. roughed or jagged surface), hooks, barbs, or other fixation elements or devices may be used with any of the filter embodiments described herein.

In some embodiments the material capture structure can include a plurality of filter elements. In some embodiments the material capture structure can extend tautly across the area between the support members. In some embodiments the material capture structure can have a basket configuration or windsock configuration.

The support members can be configured to collapse and expand with natural vessel movements while maintaining constant apposition with the vessel wall. One result is that the support members shape and size track to vessel movements. As a result, the filter density and capacity of embodiments of the present invention remain relatively independent of changes in vessel size. Moreover, the self centering aspect of the support structure ensures the filtration device provides uniform filtration across the vessel diameter. As such, embodiments provide generally constant filtration capacity of the device across the entire vessel lumen and during vessel contractions and expansions.

Uniform filter capacity is a significant improvement over some conventional devices. Conventional devices typically have a filter capacity that varies radially across a lumen. The radial variation in filter capacity usually results from the fact that conventional filtration elements have a generally wider spacing at the periphery of the lumen and closer spacing along the central lumen axis. The result is that larger emboli can escape along the lumen periphery. During vessel expansions and contractions, the radial variations in filter capacity are exacerbated in conventional devices.

Another advantage of some embodiments is that when released from a constrained state (i.e., within a delivery sheath), the device assumes a pre-determined form with the support frame self centering the device in the vessel. The support frame exerts atraumatic radial force against the vessel wall to prevent or minimize device migration. In some embodiments, radial forces generated by the support frame work in cooperation with an optionally textured support frame surface and hooks, barbs or other fixation devices to secure the device within the vessel. Hooks, barbs, or other fixation devices or elements may be used as an added precaution against migration of the filtering device while in a lumen. When device retrieval is initiated, the uniformly collapsible form of the support frame causes the support frame to pull away from the vessel wall as the device is being re-sheathed. The movement of the support frame away from the vessel wall facilitates the atraumatic removal of the device from the vessel wall. Additionally, in those embodiments having hooks, barbs or other fixation devices or elements, elongate member movement during retrieval also facilitates withdrawal of the fixation elements from the lumen wall.

Additional embodiments of the present invention may include a retrieval feature on one or both ends of the device. The use of retrieval features on both ends of the device allows deployment, repositioning and removal of the device to be accomplished from either end of the device. As a result, the use of retrieval features on both ends of the device enables both antegrade or retrograde approaches to be used with a single device. The retrieval feature may be integral to another structural member or a separate component. In some embodiments, the retrieval feature is collapsible and may have a curved shape or a generally sinusoidal shape.

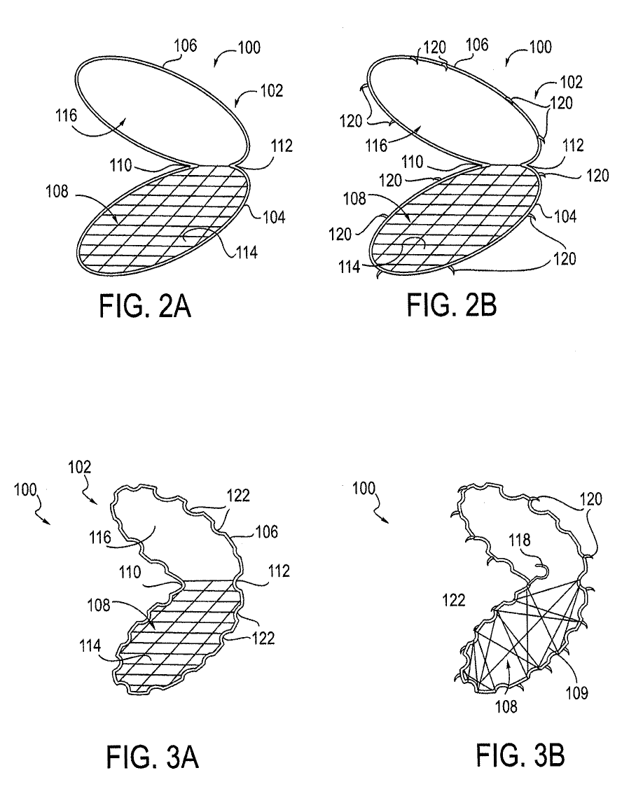

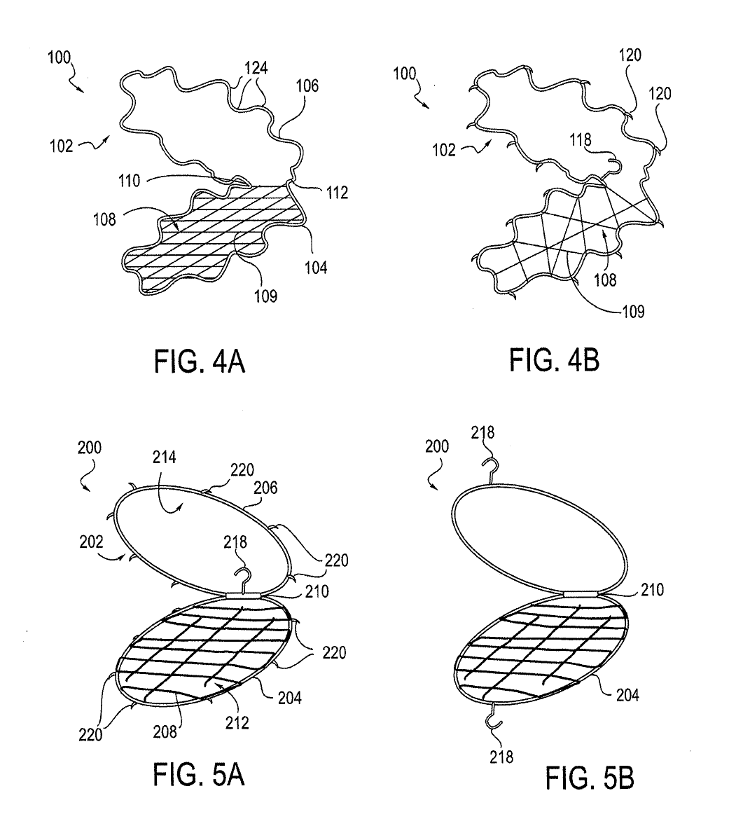

In some embodiments the filters disclosed herein have a structure resembling two-partial loops or semi-circular structures (FIGS. 2A-2B, 3A-3B, and 4A-4B). The embodiments of filters resembling two-partial loops can be made out of a single wire or assembled from multiple wires or pieces. In some embodiments the material capture structure can extend between one or both of the partial loop or semi-circular structures. The material capture structure 108 can extend within the partial loop structure in a grid pattern (FIGS. 2A, 2B, 3A, and 4A) or can extend between bends, segments, steps, zigzags, or sinusoidal bends in the partial loop structure (FIGS. 3B and 4B).

In some embodiments the partial loop structure has a smooth circular or elliptical structure as illustrated in FIGS. 2A-2B. In some embodiments the partial loop structure has a segmented configuration as in FIGS. 3A-3B. In some embodiments the partial loop structure has a sinusoidal shape with a plurality of bends or inflection points as shown in FIGS. 4A-4B. In some embodiments the partial loop structures can be mixed, such as combining a smooth partial loop structure with a segmented or sinusoidal partial loop structure.

A filter retrieval feature can be placed at various places on the partial loops. In some embodiments the retrieval feature can be in a middle area adjacent to where the two partial loops or semi-circular structures meet as shown in FIGS. 2B, 3B, and 4B. In some embodiments the filter retrieval feature can be on one end of either of the partial loops.

The embodiments of filters with two-partial loops can optionally include anchors projecting form the partial loop structures to atraumatically secure the filter within a lumen. In some embodiments the segmented configuration (FIGS. 3A-3B) or sinusoidal bend configuration (FIGS. 4A-4B) provides additional friction between the filter and the lumen wall to hold the filter in place.

In some embodiments filters are disclosed having a first full loop and additional support structure. Examples of filters with a first full loop and additional support structure are illustrated in FIGS. 5, 6, 8, 11, and 13-22.

In some embodiments the additional support structure includes a second loop connected directly or indirectly to the first loop structure. Examples of such embodiments are shown in FIGS. 5A-5B, 6A-6B, 8A-8B, 11, 14, 15, and 21. The second loop structure can be directly connected to the first loop structure with a connection point or mechanical structure as shown in FIGS. 5A-5B. Examples of the connection point include a hinge, crimp, a tube, wire, weld, or other mechanical structure. In some embodiments the connection point fixes the first loop structure relative to the second loop structure. In some embodiments the connection point allows for relative movement between the first loop structure and second loop structure.

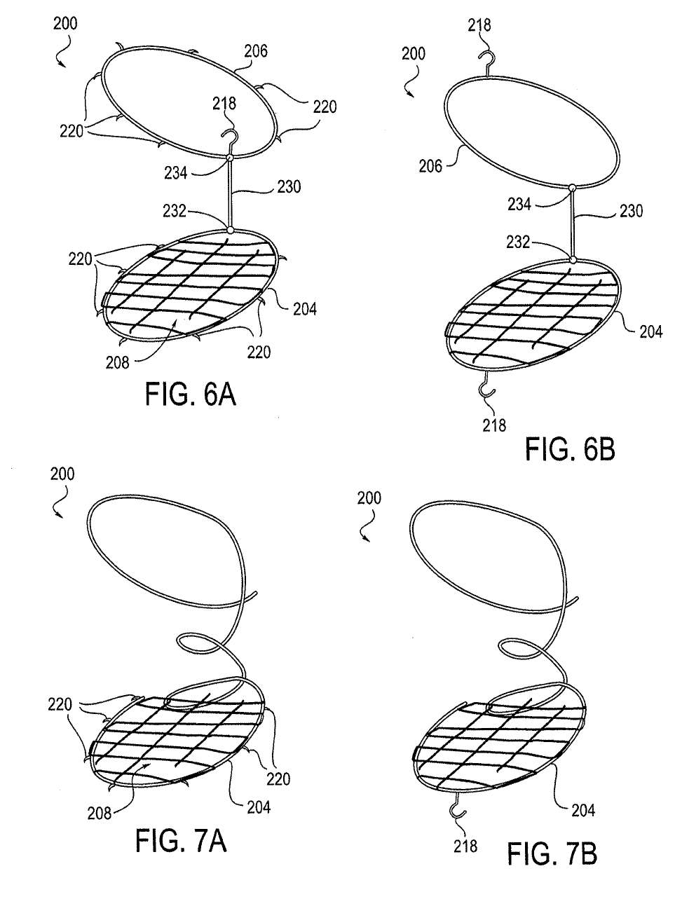

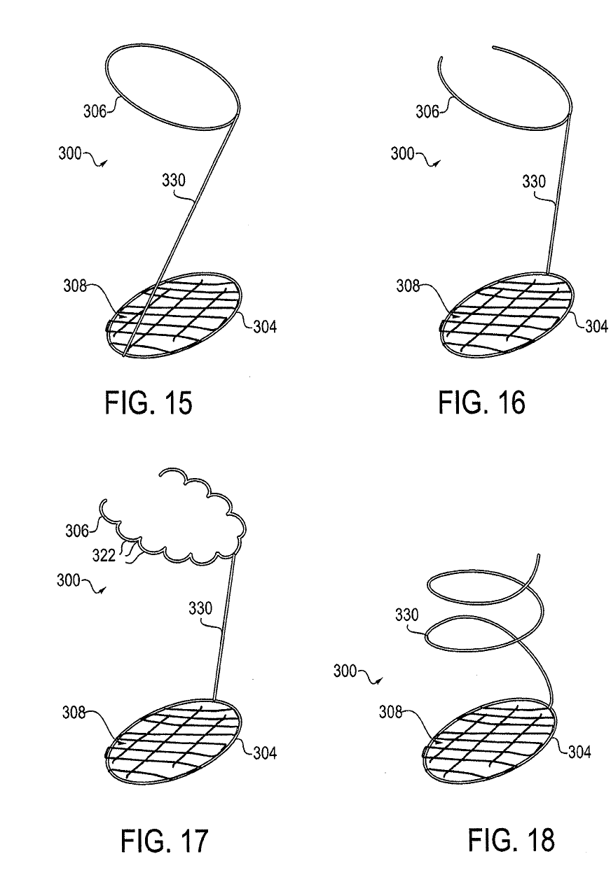

In some embodiments the first loop structure and second loop structure are connected by a third support member. Examples of embodiments with a third support member include FIGS. 6A-6B, 11, 14, and 15. The third support member can include an elongate configuration, coiled configuration, or other shapes. The loops can be fixed at the connection point to the third member or capable of relative movement about the connection point. In some embodiments the loops are connected by a single elongate support member as shown in FIGS. 6A-6B. In some embodiments the loops are connected by two support members as shown in FIG. 14. In some embodiments the third support member is connected to the first loop structure on one side and connected to the second loop structure on an opposing side (FIG. 15) such that when the filter is in a lumen the third support member extends from one side of the lumen to the other side of the lumen.

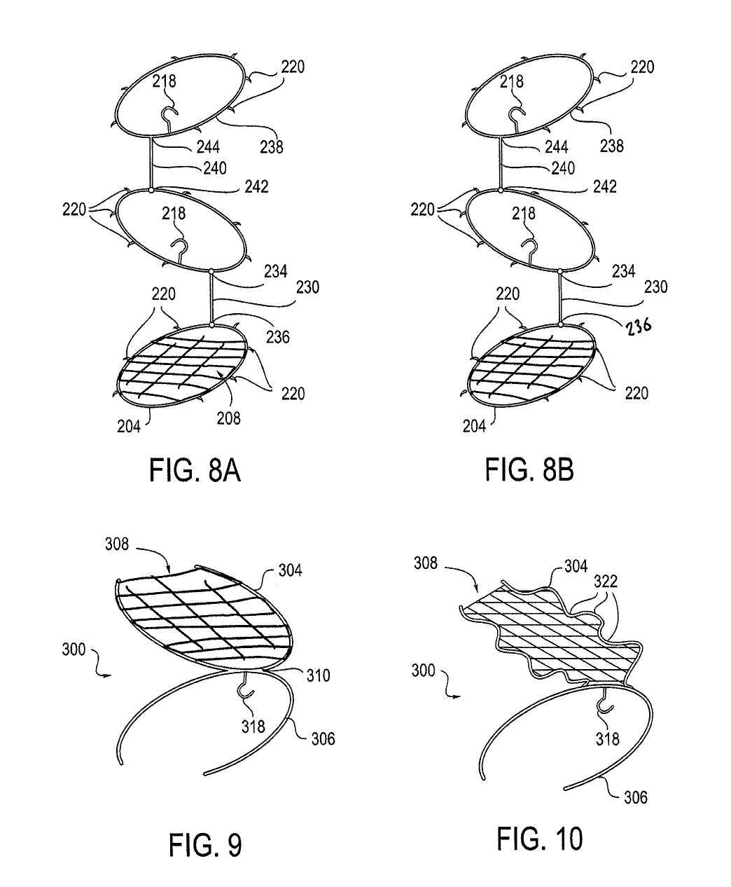

In some embodiments the additional support can be two or more additional loop structures. The additional support structure can include two loops to result in a filter with three or more loop structures. Examples of filters having three loops are illustrated in FIGS. 8A-8B. A material capture structure can extend between any or all of the loop structures in the filter.

In some embodiments a coiled support member can be attached to the loop structure as illustrated in FIG. 18.

In some embodiments the additional support structure includes an arched or semi-circular support member directly or indirectly attached to the first support member. Examples of arched or semi-circular support members attached to the first loop structure are shown in FIGS. 13, 16, 17, 19, 20, and 22. The arched or semi-circular support members can be attached directly or indirectly to the first loop structure. A third support member can connect the first loop structure with the arched or semi-circular support member. The third support member can include an elongate configuration, coiled configuration, or other shapes. The first loop and arched support member can be fixed at the connection point to the third member or capable of relative movement about the connection point.

In some embodiments any of the loop structures disclosed herein can have a plurality of segments. An example of a loop with a plurality of segments is illustrated in FIG. 21.

In some embodiments any of the loop structures disclosed herein can have a plurality of bends or inflection points. An example of a loop with a plurality of bends/inflection points is illustrated in FIG. 22.

In some embodiments filters with two arched or semi-circular structures are disclosed. Examples of filters with two-arched or semi-circular structures are illustrated in FIGS. 9, 10, and 12. The arched structures have additional flexibility to conform within the lumen with the free ends.

The first arch structure can be formed from a first support member and the second arch structure can be formed from a second support member. The first support member can be fixed directly or indirectly to the second support member. The first support member and second support member can be connected to each other using a weld, crimp, or other mechanical means. The first support member and second support member can be fixed relative to each other at the connection point or the connection point can allow for relative movement between the first support member and second support member.

In some embodiments the connection point between the first support member and second support member can be formed at approximately the midpoint of the support members. In some embodiments the connection point between the first support member and second support member can be formed at a position that is between the midpoint and the end of the support member.

Either or both of the arched structures can include a plurality of bends, segments, or inflection points in the support members. Examples of bends and inflection points are shown in FIGS. 10, 12, and 17. The bends can serve as filter attachment points for the material capture structure. The bends can also improve apposition between the support member and lumen wall.

The filters disclosed herein can be made out of any biocompatible material. Examples of biocompatible materials include shape memory materials, biocompatible polymers, biodegradable polymers, and biocompatible materials. In one embodiment, the support frame is formed from MRI compatible materials. The support frame preferably contains no sharp bends or angles to produce stress risers that may lead to fatigue issues, vessel erosion, and facilitate device collapse.

Examples of suitable shape memory alloy materials include, for example, copper-zinc-aluminium, copper-aluminum-nickel, and nickel-titanium (NiTi or Nitinol) alloys. Shape memory polymers may also be used to form components of the filter device embodiments of the present invention. In general, one component, oligo(e-caprolactone) dimethacrylate, furnishes the crystallizable “switching” segment that determines both the temporary and permanent shape of the polymer. By varying the amount of the comonomer, n-butyl acrylate, in the polymer network, the cross-link density can be adjusted. In this way, the mechanical strength and transition temperature of the polymers can be tailored over a wide range. Additional details of shape memory polymers are described in U.S. Pat. No. 6,388,043 which is incorporated herein by reference in its entirety. In addition, shape memory polymers could be designed to degrade. Biodegradable shape memory polymers are described in U.S. Pat. No. 6,160,084 which is incorporated herein by reference in its entirety.

Biodegradable polymers may also be used to form components of embodiments of the filter devices disclosed herein. For example, polylactide (PLA), a biodegradable polymer, has been used in a number of medical device applications including, for example, tissue screws, tacks, and suture anchors, as well as systems for meniscus and cartilage repair. A range of synthetic biodegradable polymers are available, including, for example, polylactide (PLA), polyglycolide (PGA), poly(lactide-co-glycolide) (PLGA), poly(e-caprolactone), polydioxanone, polyanhydride, trimethylene carbonate, poly(β-hydroxybutyrate), poly(g-ethyl glutamate), poly(DTH iminocarbonate), poly(bisphenol A iminocarbonate), poly(ortho ester), polycyanoacrylate, and polyphosphazene. Additionally, a number of biodegradable polymers derived from natural sources are available such as modified polysaccharides (cellulose, chitin, dextran) or modified proteins (fibrin, casein). The most widely used compounds in commercial applications include PGA and PLA, followed by PLGA, poly(e-caprolactone), polydioxanone, trimethylene carbonate, and polyanhydride. Additional polymers that can be used include poly(amino acids) such as poly(L-glutamate), poly(L-lysine), and poly(L-leucine). Biodegradable polyurethane based materials can also be used.

In some embodiments non-polymeric materials can be used. In some embodiments non-shape memory materials are used. For example, magnesium and other biocompatible metals can also be used.

In some embodiments the filter structures disclosed herein can be made out of a single wire or multiple pieces of wire.

A circular shaped filter can be harder to collapse then a filter having a semi-circular configuration. The filter support members and frame can be modified to improve the collapsibility of the circular shape. In some embodiments the support members can include a movable portion such as an articulation point, joint, bend, kink, or other movable section to facilitate the collapse of the filter. In some embodiments the filter can have multiple movable portions. Multiple movable portions can further improve the collapsibility of circular support members.

In some embodiments the filter can be designed to be used in veins. Veins can have large swings in the diameter. The filter can be designed to have a flexible diameter to adjust to the diameter of the vein during use.

In some embodiments the filter can be designed to be used in an artery. Arteries do not typically have a changes in the artery diameter during use. A device with a more rigid diameter or fixed diameter can be used in arteries. In some embodiments the filters can have a support frame with a fixed diameters suitable for use in an artery.

In some embodiments can be used with a tether. For example for distal protection during removal of a lesion.

In some embodiments any of the filters disclosed herein can be used as an occlusion device. The material capture structure can have a finer structure or a solid structure to act as an occlusion device. Suitable materials for an occlusion application include for example, wool, silk polymer sheets, other material suited to prevent blood flow in a lumen when extended across a lumen and the like. Embodiments of the filters disclosed herein acting as occlusion devices can be deployed and retrieved using any of the methods disclosed herein.

Any of the filter devices disclosed herein can be deployed and retrieved using minimally invasive catheter techniques. In some embodiments the material capture side of the filter is deployed first. In some embodiments the material capture side of the filter is deployed second. In some embodiments the deployed filter is captured using a retrieval feature on one of the ends of the filter.

In some embodiments the filter is captured using a retrieval feature near a midpoint or fixation point on the filter. One advantage of positioning the retrieval feature near the midpoint of the filter is that it decreases the overall length of the deployed filter in comparison to a filter having a retrieval feature at one of the ends of the filter because the retrieval feature does not contribute to the overall length of the filter. Positioning the retrieval feature near the midpoint of the filter also provides multiple retrieval feature opportunities.

Contacting the retrieval feature on the filter can cause the filter to collapse. The collapsed filter can then be retrieved through the catheter in a minimally invasive way. In embodiments having a circular support member a movable portion can be used to improve the collapsibility of the circular support member.

Various features shown in the embodiments illustrated in the figures are now discussed. FIGS. 2A-2B illustrate various features of endoluminal filter devices having two semi-circular structures. FIG. 2A shows an endoluminal filter 100. The endoluminal filter 100 includes a support frame 102 formed from a first support member 104 and a second support member 106. The first support member 104 meets the second support member 106 at points 110, 112. The first support member 104 and second support member 106 converge at points 110 and 112 but are still laterally spaced apart and thus the support members do not crossover. The first support member 104 forms an acute angle with the second support member 106 at 110 and 112. In some embodiments the acute angle is less than about 60°. In some embodiments the acute angle is less than about 45°. In some embodiments the acute angle is less than about 30°. The first support member 104 forms a more pointed structure with the second support member 106 at points 110 and 112 in comparison to the more rounded points 110, 112 illustrated in FIGS. 3A-3B. The first support member 104 has a semi-circular configuration with a material capture structure 108 within an internal area 114 defined by the first support member 104. The second support member 106 has a semi-circular configuration with an internal area 116 defined by the second support member 106. The internal area 114 of the first second support member 106 can optionally include a material capture structure.

FIG. 2B illustrates an endoluminal filter device 100 similar to the filter in FIG. 2A. The filter 100 in FIG. 2B also includes a retrieval feature 118 and anchors 120. The anchors 120 are illustrated as projections off of the support frame 102. The anchors 120 can hold the filter 100 within the lumen. The retrieval feature 118 illustrated herein are positioned such that when the filter is implanted within the vessel the retrieval feature 118 is accessible to a retrieval snare. For example, the retrieval feature 118 can be positioned so that it points away from the vessel wall towards the interior volume of the vessel.

FIGS. 3A-3B illustrate various features of endoluminal filter devices. FIG. 3A shows an endoluminal filter 100 with a support frame 102 formed from a first support member 104 and a second support member 106. The first support member 104 meets the second support member 106 at 110, 112. The first support member 104 and second support member 106 have semi-circular footprints along with curved segments 122. The curved segments 122 can provide additional strength to the support frame 102 and also improve the friction of the support frame 102 with the lumen wall. A material capture structure 108 is illustrated within an internal area 114 defined by the first support member 104. The second support member 106 has a semi-circular configuration with an internal area 116 defined by the second support member 106. The internal area 116 of the first second support member 106 can optionally include a material capture structure.

FIG. 3B illustrates an endoluminal filter device 100 similar to the filter in FIG. 3A. The filter 100 in FIG. 3B also includes a retrieval feature 118 and anchors 120. The anchors 120 are illustrated as projections off of the support frame 102. The material capture structure 108 has filaments 109 extending between the curved segments 122.

FIGS. 4A-4B illustrate various features of endoluminal filter devices. FIG. 4A shows an endoluminal filter 100 with a support frame 102 formed from a first support member 104 and a second support member 106. The first support member 104 and the second support member 106 have sinusoidal bends 124. The sinusoidal bends 124 can also be described as having a plurality of bends or a plurality of inflection points. The sinusoidal frame configuration can provide additional strength to the support frame 102 and also improve the friction of the support frame 102 with the lumen wall. A material capture structure 108 is illustrated within an internal area 114 defined by the first support member 104.

FIG. 4B illustrates an endoluminal filter device 100 similar to the filter in FIG. 4A. The filter 100 in FIG. 4B also includes a retrieval feature 118 and anchors 120. The anchors 120 are illustrated as projections off of the support frame 102. The material capture structure 108 has filaments 109 extending between the sinusoidal bends 124.

FIGS. 5A-6B illustrate various features of endoluminal filter devices having two loop configurations. FIG. 5A illustrates an endoluminal filter 200. The support frame 202 includes a first support member 204 forming a first loop and a second support member 206 forming a second loop. The first support member 204 and second support member 206 are joined together by connector 210. The connector 210 is illustrated with a clamp or crimp type structure. Other structures are also possible to secure the support members together such as a hinge, welding, mechanical tie, etc. A material capture structure 208 spans the internal area 212 of the first loop structure formed by the first support member 204. Anchors 220 project from the first support member 204 and second support member 206 to securely hold the filter 200 within the lumen. A retrieval feature 218 is located on the connector 210. The retrieval features 218 illustrated herein are positioned such that when the filter is implanted within the vessel the retrieval feature 218 is accessible to a retrieval snare. For example, the retrieval feature 218 can be positioned so that it points away from the vessel wall towards the interior volume of the vessel.

The endoluminal filter 200 illustrated in FIG. 5B has a similar structure to the filter in FIG. 5A with the retrieval feature located at a different position. In FIG. 5B there are two retrieval features 218, with one retrieval feature 218 located on the first support member 204 and the second retrieval feature 218 located on the second support member 206.

FIGS. 6A-6B illustrate various features of endoluminal filter devices. FIG. 6A illustrates a filter 200 with a two loop structure. The first support member 204 forms a first loop structure and the second support member 206 forms a second loop structure. The loop structures are connected by a third support member 230. The third support member 230 connects to the first support member 204 at point 232 and connects to the second support member 206 at point 234. The third support member 230 is illustrated with a straight elongated shape; however, other shapes and configurations are also possible such as a jagged or coiled shape. The retrieval feature 218 is connected to the second support member 206 near the point 234.

The endoluminal filter 200 illustrated in FIG. 6B has a similar structure to the filter in FIG. 6A with the retrieval feature located at a different position. In FIG. 6B there are two retrieval features 218, with one retrieval feature 218 located on the first support member 204 and the second retrieval feature 218 located on the second support member 206.

FIGS. 7A-7B illustrate various features of endoluminal filter devices formed from a coiled support member. The first support member 204 forms a semi-circular section with the material capture structure 208 within the semi-circular section. Anchors 220 project from the support member 204. Instead of forming a closed loop the first support member 204 has a coiled structure. The coiled structure can further secure the filter 200 within a lumen. FIG. 7B illustrates a similar filter to the filter shown in FIG. 7B but with a retrieval feature 218 on the first support member 204. Although the coils in FIGS. 7A and 7B are illustrated with a non-uniform structure, a uniform coiled structure can also be used. In some embodiments the coil can include major and minor turns or a combination of major and minor turns. In some embodiments a coiled structure can be used as a retrieval feature.

FIGS. 8A-8B illustrate various features of endoluminal filter devices. FIGS. 8A-8B are similar to FIGS. 6A-6B but with a third loop structure. In FIG. 8A the filter 200 has a third loop structure 238 connected to the second support member 206 by a connector 240. The connector 240 is attached to the second support member 206 at point 242 and is attached to the third loop structure at point 244. The filter 200 includes retrieval features 218 on the second support member 206 and the third loop structure 238. Anchors 220 project from the first support member 204, second support member 208, and third loop structure 238. FIG. 8B illustrates a filter 200 with the retrieval feature 218 attached to the first support member 204.

FIG. 9 illustrates an endoluminal filter 300 in accordance with an embodiment. The filter 300 includes a first support member 304 with an arched or semi-circular configuration. A material capture structure 308 extends between the walls of the first support member 304. The first support member 304 is connected to a second support member 308 at connection 310. The second support member 308 has an arched or semi-circular configuration. A retrieval feature 318 is on the filter 300 adjacent to the connector 310. The retrieval feature 318 illustrated herein is positioned such that when the filter is implanted within the vessel the retrieval feature 318 is accessible to a retrieval snare. For example, the retrieval feature 318 can be positioned so that it points away from the vessel wall towards the interior volume of the vessel.

FIG. 10 illustrates a filter 300 in accordance with an embodiment. The filter 300 includes a first support member 304 with an arched or semi-circular configuration and a plurality of bends 322 forming a sinusoidal shaped perimeter. A material capture structure 308 extends between the walls of the first support member 304. The first support member 304 is connected to a second support member 308 at connection 310. The second support member 308 has an arched or semi-circular configuration. A retrieval feature 318 is on the filter 300 adjacent to the connector 310.

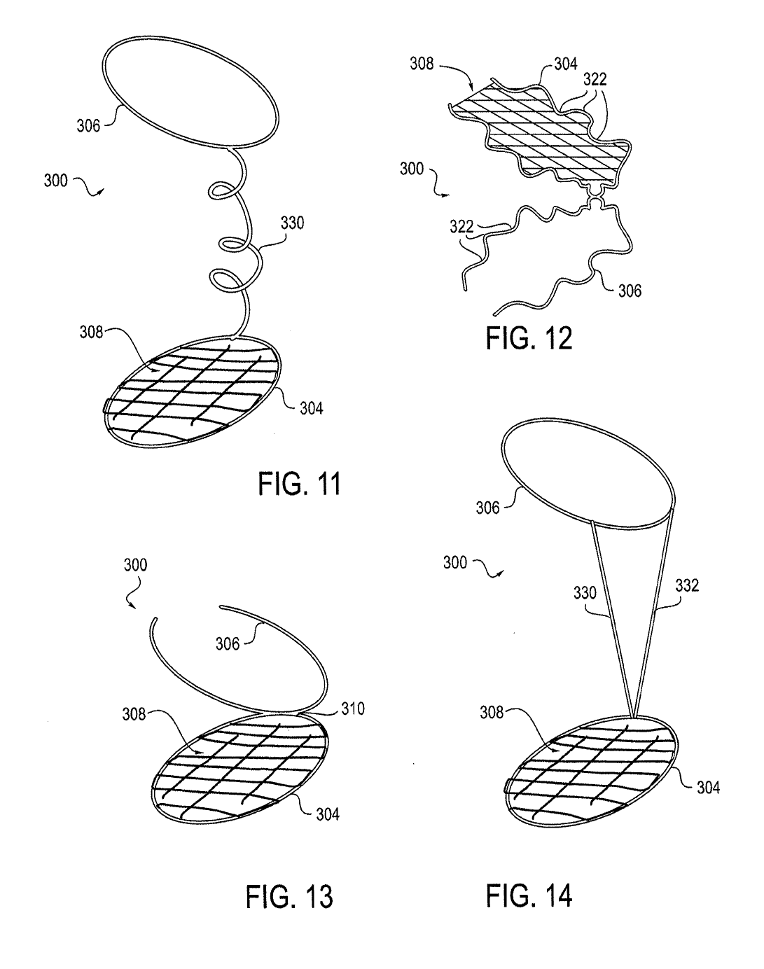

FIG. 11 illustrates a filter 300 in accordance with an embodiment. The filter 300 includes a first support member 304 with a loop structure. A material capture structure 308 extends within the internal area of the loop structure. A second support member 306 has a loop structure. The second support member 306 is connected to the first support member 304 by a third support member 330 having a coiled configuration. While the support member 330 is illustrated with a coiled configuration in FIG. 11, other configurations are possible such as a straight connecting member (FIGS. 15, 16, and 17), more than one connecting element (FIG. 14), a uniform coiled structure, or may contain more than one undulation (FIG. 12).

FIG. 12 illustrates a filter 300 in accordance with an embodiment. The filter 300 includes a first support member 304 with an arched or semi-circular configuration and a plurality of bends 322 forming a sinusoidal shaped perimeter. A material capture structure 308 extends between the walls of the first support member 304. The first support member 304 is connected to a second support member 308 at connection 310. The second support member 308 has an arched or semi-circular configuration and a plurality of bends 322 forming a sinusoidal shaped perimeter.

FIG. 13 illustrates a filter 300 in accordance with an embodiment. A first support member 304 forms a loop structure with a material capture structure 308 within the area defined by the loop structure. The first support member 304 is connected to a second support member 306 at connection 310. The second support member 306 is configured in an arched or semi-circular configuration.

FIG. 14 illustrates a filter 300 in accordance with an embodiment. The filter 300 includes a first support member 304 with a loop structure. A material capture structure 308 extends within the internal area of the loop structure. A second support member 306 has a loop structure. The second support member 306 is connected to the first support member 304 by a third support member 330 and a fourth support member 332.

FIG. 15 illustrates a filter 300 in accordance with an embodiment. The filter 300 includes a first support member 304 with a loop structure. A material capture structure 308 extends within the internal area of the loop structure. A second support member 306 has a loop structure. The second support member 306 is connected to the first support member 304 by a third support member 330 at an opposing side of the second support member 306 from where the third support member 330 is connected to the first support member 304.

FIG. 16 illustrates a filter 300 in accordance with an embodiment. A first support member 304 forms a loop structure with a material capture structure 308 within the area defined by the loop structure. The first support member 304 is connected to a second support member 306 by a third support member 330. The second support member 306 is configured in an arched or semi-circular configuration.

FIG. 17 illustrates a filter 300 in accordance with an embodiment. A first support member 304 forms a loop structure with a material capture structure 308 within the area defined by the loop structure. The first support member 304 is connected to a second support member 306 by a third support member 330. The second support member 306 is configured in an arched or semi-circular configuration and a plurality of bends 322 forming a sinusoidal shaped perimeter.

FIG. 18 illustrates a filter 300 in accordance with an embodiment. The filter 300 has a first support member 304 in a loop structure. A material capture structure 308 spans the area within the loop structure formed by the first support member 304. A third support member 330 is connected to the first support member 308 with a coiled configuration. The coiled configuration of the third support member 330 can be configured to contact the lumen and hold the filter 300 in place.

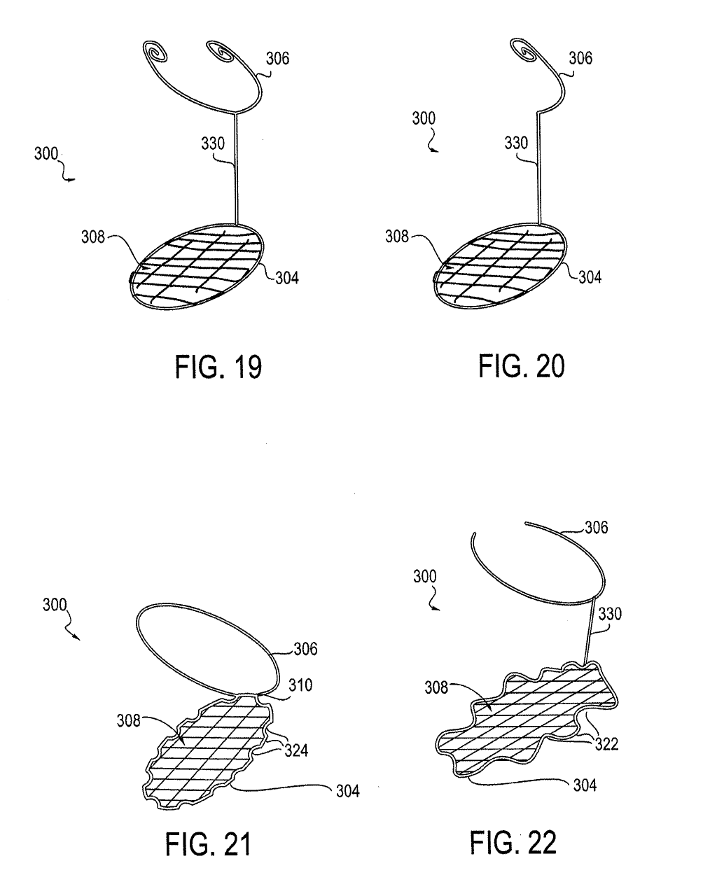

FIG. 19 illustrates a filter 300 in accordance with an embodiment. A first support member 304 forms a loop structure with a material capture structure 308 within the area defined by the loop structure. The first support member 304 is connected to a second support member 306 by a third support member 330. The second support member 306 is configured in an arched or semi-circular configuration having curled ends.

FIG. 20 illustrates a filter 300 in accordance with an embodiment. A first support member 304 forms a loop structure with a material capture structure 308 within the area defined by the loop structure. The first support member 304 is connected to a second support member 306 by a third support member 330. The second support member 306 is configured in a half-circular configuration with a curled end. The second support member 306 is configured to span the lumen diameter and hold the filter in place within the lumen.

FIG. 21 illustrates a filter 300 in accordance with an embodiment. A first support member 304 forms a loop structure with a material capture structure 308 within the area defined by the loop structure. The first support member 304 includes a plurality of curved segments 324. The second support member 306 forms a loop structure. The first support member 304 is connected to the second support member 306 at connection 310.

FIG. 22 illustrates a filter 300 in accordance with an embodiment. A first support member 304 forms a loop structure with a material capture structure 308 within the area defined by the loop structure. The first support member 304 includes a plurality of bends 322 forming a sinusoidal shaped perimeter. The first support member 304 is connected to a second support member 306 by a third support member 330. The second support member 306 is configured in an arched or semi-circular configuration.

Various configurations for the support member are illustrated in the figures. In some embodiments any of the different support member configurations can be used with the first support member and second support member described herein. For example a coiled configuration (FIG. 11), an irregularly coiled configuration, a uniform tightly coiled structure, a straight connecting member (FIGS. 15, 16, and 17), more than one connecting element (FIG. 14), or an undulating support member (FIG. 12) can be used with any of the first and second support member structures described herein.

Various structures are disclosed herein for joining the various support members described herein. The embodiments of support members and filters described herein can include any of the embodiments of joining structures described herein. In some embodiments the support members are integrally formed (e.g. FIGS. 2A-4B). In some embodiments the support members are connected using a connector, such as connector 210. The illustrated connector 210 has a tubular shape, although other connector shapes can be used. In some embodiments the support member can move relative to the connector 210, such as by sliding. In some embodiments the support members are connected by a connection point, such as points 232, 234. The connection point can be fixedly or rigidly connected to the support member. In some cases the connection point can allow for relative movement between the point and the support member. In some embodiments the support members are joined by welding. In some embodiments a flexible joint is used.

Various configurations can be used for the support frame or member that doesn't have the material capture structure. In some embodiments any of the support frames or members that do not have the material capture structure can be mixed and matched with any of the support members or loops having material capture structures. A closed support structure, such as those illustrated in FIGS. 11, 14, 15, and 21, can be used with any of the material capture structures described herein. An open structure with regular curvature and no terminal feature, such as those illustrated in FIGS. 13 and 16, can be used with any of the material capture structures described herein. A structure with a curved terminal feature, such as those illustrated in FIGS. 19 and 20, can be used with any of the material capture structures described herein. A structure with an undulating element, such as the structure illustrated in FIG. 17, can be used with any of the material capture structures described herein.

Any of the manufacturing techniques disclosed in U.S. Provisional Patent Application No. 62/090,580, filed on Dec. 11, 2014, and tilted “ENDOLUMINAL FILTER DESIGN VARIATIONS,” can be used to make the filter structures disclosed herein. In some embodiments the filters can be manufactured via three-dimensional (3D) printing. In some embodiments the filters can be manufactured by cutting the frame shape out of a tubular structure, such as a Nitinol tube. In some embodiments the frame support structure can be made out of a micro-truss structure.

Material Capture Structures

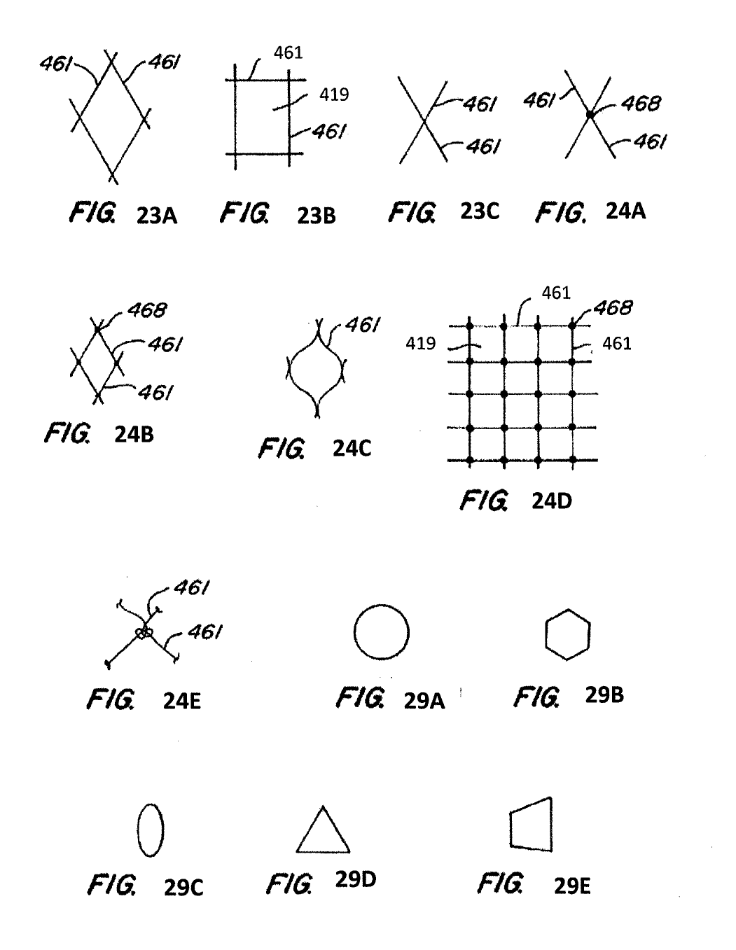

The material capture structures illustrated in FIGS. 23A-34F can be used in any of the embodiments of endoluminal filters described herein. The material capture structure can be selected based on the desired application for the filter. For example if the filter is for arterial and/or distal protection then a fine material capture structure could be used. In another example, if the filter is for embolic protection then the material capture structure can be occlusive.

In some embodiments, the material capture structure contains a number of filter cells. Filter cells may be formed in a number of different ways and have a number of different shapes and sizes. The shape, size and number of filter cells in a specific filter may be selected based on the use of a particular filter. For example, a filter device of the present invention configured for distal protection may have a filter cell size on the order of tens to hundreds of microns to less than 5 millimeters formed by a selecting a filter material with a pore size (FIGS. 24A, 24B) suited to the desired filtration level. In other applications, the filter cell may be formed by overlapping (i.e., joined or crossed without joining) filaments to form cells that will filter out debris in a lumen above a size of 2 mm. Various other filter sizes and filtration capacities are possible as described herein.

Intersecting filaments (FIG. 23C) may be used to form diamond shaped filter cells (FIG. 23A), as well as rectangular shaped filter cells 419 (FIG. 23B). Multiple strand patterns may also be used such as the three strand 461a, 461b and 461c array illustrated in FIG. 26B. Intersecting filaments may also be knotted, tied or otherwise joined 468 (FIGS. 24A and 24E). Intersecting filaments may form the same or different filter cell shapes such as, for example, an elongated oval in FIG. 24C, one or more joined diamonds as in FIG. 24B and an array of joined polygons as in FIG. 14D. In one embodiment, a filter cell is defined by at least three intersecting filaments 461. The filter element 461 may be formed from any of a wide variety of acceptable materials that are biocompatible and will filter debris. For example, filaments, lines and strands described herein may be in the form of a multifilament suture, a monofilament suture a ribbon, a polymer strand, a metallic strand or a composite strand. Additionally, filaments, lines and strands described herein may be formed from expanded polytetrafluoroethylene (ePTFE), polytetrafluoroethylene (PTFE), Poly(ethylene terephthalate) (PET), Polyvinylidene fluoride (PVDF), tetrafluoroethylene-co-hexafluoropropylene (FEP), or poly(fluoroalkoxy) (PFA), other suitable medical grade polymers, other biocompatible polymers and the like.

The joined polygons may have any of the shapes illustrated in FIGS. 29A-29F. It is to be appreciated that filter cells may have any, one or more, or hybrid combinations of shapes such as, for example, circular (FIG. 29A), polygonal (FIG. 29B), oval (FIG. 29C), triangular (FIG. 29D), trapezoidal or truncated conical (FIG. 29E).

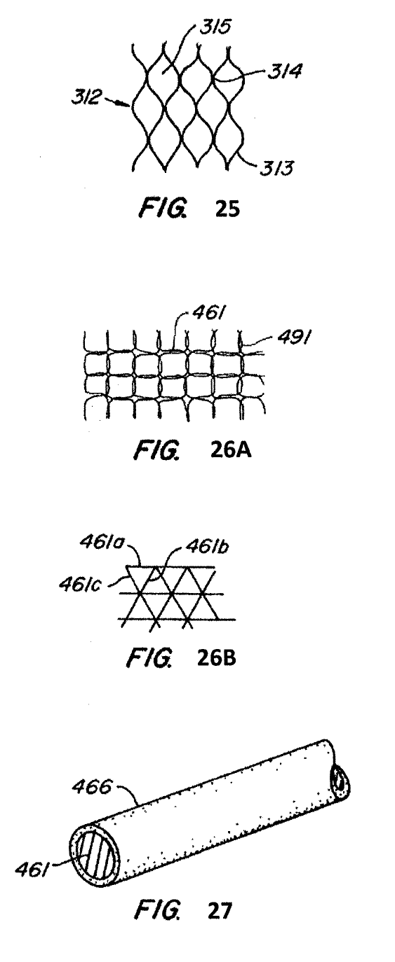

In addition, the material capture structure may have filter cells formed by extruding a material into a material capture structure. FIG. 25 illustrates an exemplary filtering structure 312 where a material is extruded into strands 313 that are joined 314 and spaced apart for form one of more filter cells 315. In one embodiment, the strands are extruded from Polypropylene material, forming diamond shaped filter cells approximately 4 mm in height and 3 mm in width.

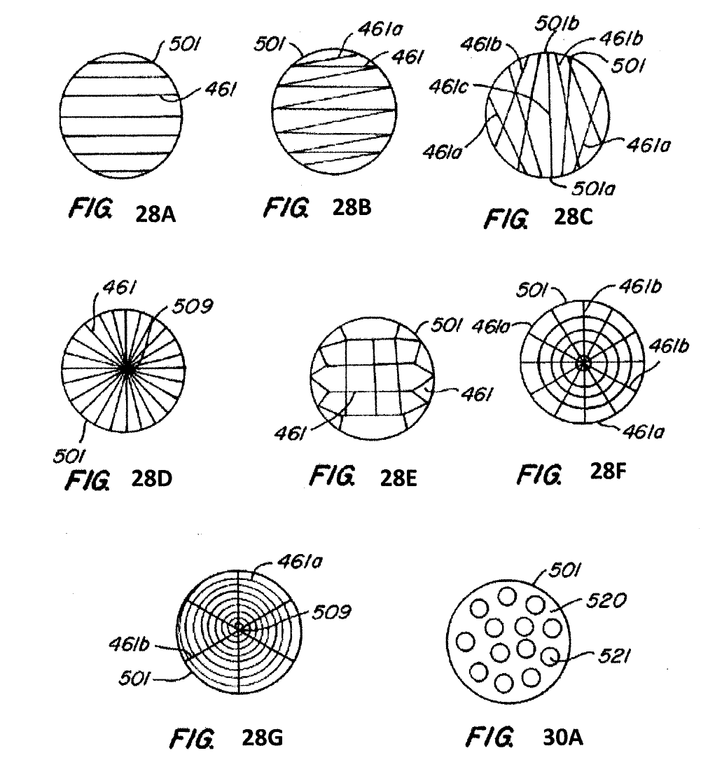

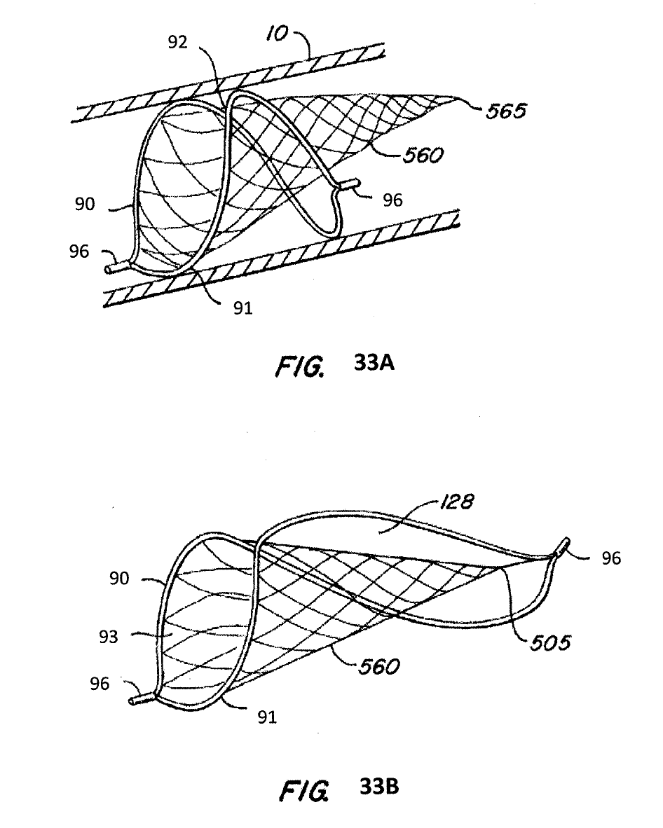

FIGS. 28A-32B illustrate several different filtering structure configurations. For simplicity of illustration, the filtering material is shown attached to a circular frame 501. It is to be appreciated that the circular frame 501 represents any of the various open loop, rounded frame or other support frames described herein. FIG. 28A illustrates a frame pattern similar to FIG. 31D. FIG. 28B adds an additional transverse filaments 461a at an angle to the filaments 461. FIG. 28C illustrates a plurality of filaments 461a extending up from the frame bottom 501a about a central filament 461c and a plurality of filaments 461b extending down from the frame top 501b about a central filament 461c. In this illustrative embodiment, the filaments 461a, b are arranged symmetrically about the central filament 461c. Other non-symmetrical configurations are possible. More than one central filament 461c may be used to form a variety of different size and shaped polygonal filter cells (e.g., FIG. 28E).

Filaments may also be arranged using a variety of radial patterns. Fr example, multiple filaments 461 may from a common point 509 out the edge of frame 501. In some embodiments, the common point is central to the frame 501 (FIG. 28D) and in other embodiments the common point 509 is in a different, non-central location. The sectors formed by the multiple filaments (FIG. 28D) may be further divided into multiple filter cell segments by winding a filament 461a about and across segment filaments 461b. In contrast to a single filament spirally out from the point 509 as in FIG. 28G, the segmented filter cells in FIG. 28F are formed by attaching single filament 461a to the segment filaments 461b.

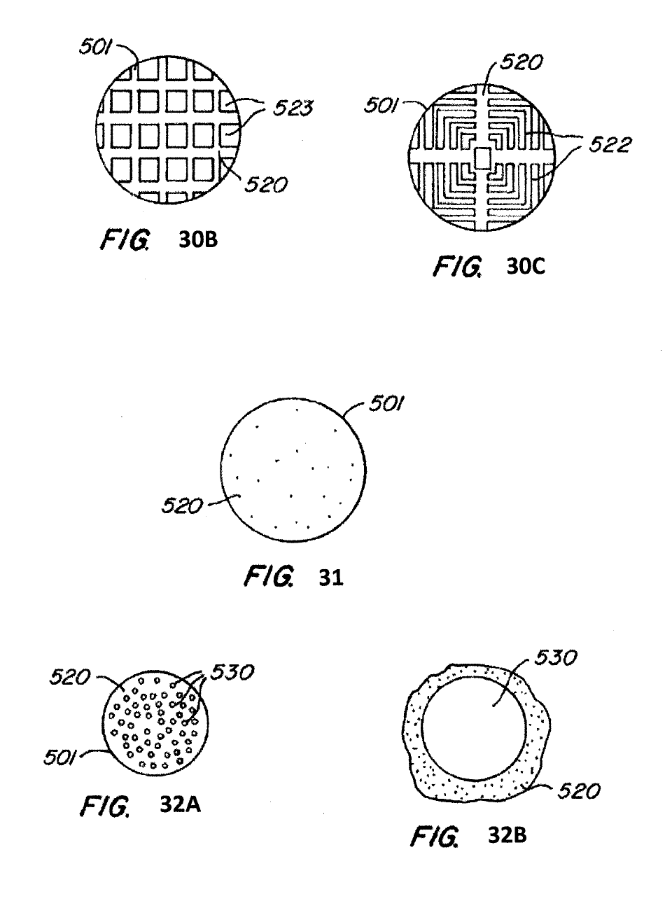

FIGS. 30A-C and FIG. 31 illustrate the use of a sheet of material 520 to form a filter structure. The material 520 may have any of a variety of shapes formed in it using any suitable process such as punching, piercing, laser cutting and the like. FIG. 30A illustrates a circular pattern 521 formed in material 520. FIG. 30B illustrates a rectangular pattern 523 formed in material 520. FIG. 30C illustrates a complex pattern 522 cut into material 522. It is to be appreciated that the material 520 may also be placed in the frame 501 without any pattern (FIG. 31). The illustrative embodiment of FIG. 31 may be useful for occluding the flow within a lumen. Suitable materials 520 for an occlusion application include for example, wool, silk polymer sheets, other material suited to prevent blood flow in a lumen when extended across a lumen and the like. Additionally, the filter material 520 may be a porous material having pores 530 (FIG. 32A). The material 520 may be selected based on the average size of individual pores 530 (FIG. 32B) depending upon the procedure or use of the filter device. For example, the material 520 may be any of the porous materials using in existing distal protection and embolic protection devices. In general, a wide variety of pore 530 sizes are available and may range from 0.010″ to 0.3″. Other pore sizes are also available depending upon the material 520 selected.

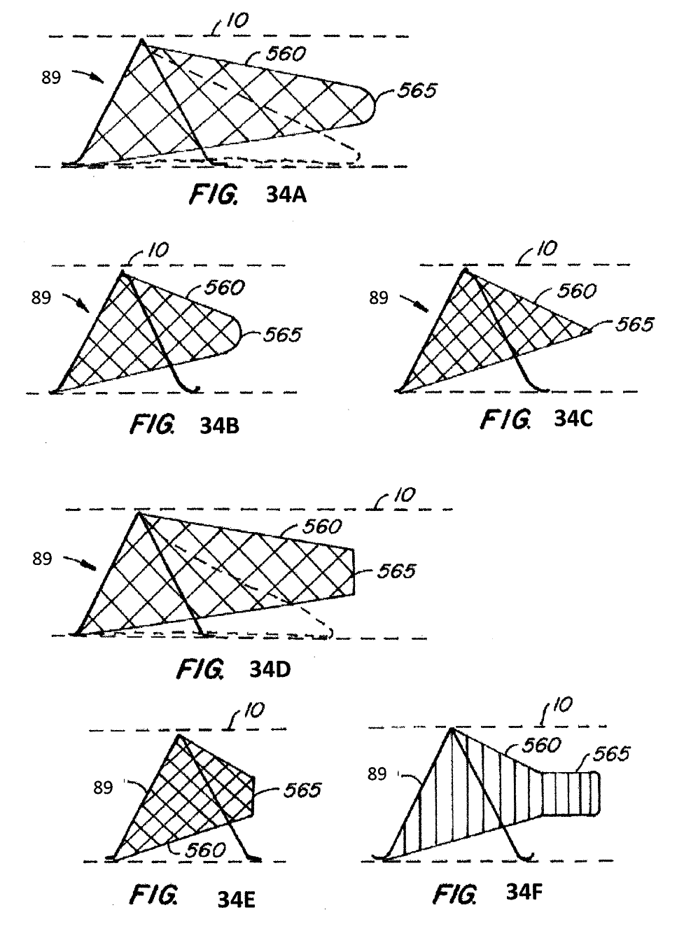

FIGS. 23-34F illustrate the use of nets or other web structures within the filtering device. The various net structure embodiments described herein are used as material capture structures within filter device embodiments of the present invention. Each of these alternative is illustrated in a support structure similar to that of device 89 in FIG. 1I and elsewhere; however, any of the alternatives can also be used with all of the embodiments of filter designs disclosed herein. When deployed within the lumen 10, the material capture structure 560 has a defined shape such as a cone with a discrete apex 565 (FIG. 33A). In this embodiment, the net structure is long enough to contact the sidewall of the lumen 10 when deployed in the lumen 10. Alternatively, the apex 565 may be attached to the end 96 to keep the net 560 in the lumen flow path and out of contact with the lumen sidewall (FIG. 33B). The net 565 may also have a rounded apex 565 (FIG. 34A) or a truncated cone (flat bottom) (FIG. 34D). Alternatively, the net 560 may a discrete apex 565 so short that it will not contact the lumen sidewall when deployed (FIG. 34B). The short net may also have a rounded apex 565 (FIG. 34B), a flat apex (FIG. 34E) or a sharp apex (FIG. 34C). In addition, the net 560 may have a compound apex 565 (FIG. 34F).

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An endoluminal filter, comprising:
a first support member having a first end and a second end;
a second support member having a first end and a second end, the second support member connected to the first support member;
a material capture structure extending within an internal area of the first support member; and
a retrieval feature engaged with the first support member or second support member.

2. An endoluminal filter of claim 1, further comprising at least one tissue anchor on the first support member or the second support member.

3. An endoluminal filter according to claim 1 wherein the retrieval feature is formed on the surface of the filter adjacent to an attachment between the first semi-circular structure and second semi-circular structure.

4. An endoluminal filter according to claim 1 wherein the first support member and the second support member are foamed from a single wire.

5. An endoluminal filter according to claim 1 wherein the first support member and second support member are made out of a shape memory material.

6. An endoluminal filter according to claim 1 wherein the first support member and second support member have smooth surfaces.

7. An endoluminal filter of claim 1, wherein the first support member forms a first semi-circular structure, the second support member forms a second semi-circular structure, and the first end and second end of the second support structure are attached to the first end and second end of the first support member.

8. An endoluminal filter according to claim 7 wherein the first support member has a sinusoidal pattern forming the first semi-circular structure and/or the second support member has a sinusoidal pattern forming the second semi-circular structure.

9. An endoluminal filter according to claim 7 wherein the first support member and/or second support member includes a plurality of inflection points or bends.

10. An endoluminal filter according to claim 7 wherein the first semi-circular structure and/or second semi-circular structure has an elliptical shape.

11. An endoluminal filter according to claim 7 wherein the first support member and second support member form an acute angle at each of an intersection between the ends of the first support member and second support member.

12. An endoluminal filter of claim 1, wherein the first support member forms a first loop structure.

13. An endoluminal filter of claim 12 wherein the first support member further comprises a movable portion.

14. An endoluminal filter of claim 12 wherein the second support member forms a second loop.

15. An endoluminal filter of claim 14 the second support member further comprising a movable portion.

16. An endoluminal filter of claim 14 wherein the second support member is attached to the first support member.

17. An endoluminal filter of claim 14 further comprising a third support member having a first end attached to the first loop structure and a second end attached to the second loop structure.

18. An endoluminal filter of claim 17 wherein the third support member has a coiled structure.

19. An endoluminal filter of claim 17 wherein the retrieval feature is located where the third support member attaches to the first loop structure or the second loop structure.

20. An endoluminal filter of claim 17 further comprising a third loop structure and a fourth support member having a first end attached to the third loop structure and a second end attached to the second loop structure.

21. An endoluminal filter of claim 12 wherein the second support member comprises a semi-circular structure.

22. An endoluminal filter of claim 21 wherein the second support member has a sinusoidal pattern forming the semi-circular structure or a plurality of bends or inflection points.

23. An endoluminal filter of claim 12 wherein the first support member has a sinusoidal pattern in the first loop structure or a plurality of bends or inflection points in the first loop structure.

24. An endoluminal filter of claim 1, wherein the first support member forms a first semi-circular structure, the second support member forming a second semi-circular structure, the first support member attached to the second support member at a portion besides the first end or second end of the first support member or second support.

25. An endoluminal filter of claim 24 further comprising a third support member having a first end attached to the first semi-circular structure and a second end attached to the second semi-circular structure.

26. An endoluminal filter of claim 25 wherein the third support member has a coiled structure.

27. An endoluminal filter of claim 24 wherein the first support member has a sinusoidal pattern in the first semi-circular structure or a plurality of bends or inflection points in the first semi-circular structure.

28. A method of positioning a filter within a lumen, comprising:
advancing a sheath containing the filter of claim 1 through the lumen;
deploying a portion of the filter of claim 1 from the sheath into the lumen to engage the lumen wall while maintaining substantially all of the material capture of the filter within the sheath; and
deploying the material capture structure of the filter of claim 1 from the sheath to a position across the lumen.

29. The method according to claim 28 further comprising:
maneuvering a snare towards the filter in the same direction used during the advancing step; and engaging the snare with a filter retrieval feature positioned against a wall of the lumen.

30. The method according to claim 28 further comprising:
maneuvering a snare towards the filter in the opposite direction used during the advancing step; and engaging the snare with a filter retrieval feature positioned against a wall of the lumen.

31. The method according to claim 28 further comprising:
deploying the filter retrieval feature from the sheath after the deploying the material capture structure step.

32. The method according to claim 28 further comprising:
deploying a filter retrieval feature from the sheath before the deploying the material capture structure step.

33. The method of positioning a filter within a lumen according to claim 28 wherein the deploying a portion of the filter step further comprising engaging the lumen wall with a radial force generated by the filter.
